(19) United States
(12) Patent Application Publication
(10) Pub. No.: US 2026/0114920 A1
(43) Pub. Date: Apr. 30, 2026

(54) CARDIAC VALVE LEAFLET CUTTING DEVICE

(71) Applicant: Montreal Heart Institute, Montreal (CA)

(72) Inventor: Walid Ben Ali, Montreal (CA)

(21) Appl. No.: 19/373,212

(22) Filed: Oct. 29, 2025

Related U.S. Application Data
(60) Provisional application No. 63/714,481, filed on Oct. 31, 2024.

(51) Int. Cl. A61B 18/14; A61B 18/00; A61B 18/18; A61B 18/20

(52) U.S. Cl. CPC A61B 18/1492; A61B 18/1442; A61B 18/1815; A61B 18/20; A61B 2018/00369; A61B 2018/00601; A61B 2018/1452

(57) ABSTRACT

The present disclosure relates to devices, systems, and methods for cutting leaflets in cardiac valves including mitral, aortic, and tricuspid valves.

### CROSS REFERENCE TO RELATED APPLICATION

This application claims the benefit of U.S. Provisional Patent Application No. 63/714,481, filed on Oct. 31, 2024, the entire contents of which are hereby incorporated by reference herein.

### FIELD

The present disclosure relates to devices, systems, and methods for cutting leaflets in cardiac valves including mitral, aortic, and tricuspid valves.

### BACKGROUND

Heart valve diseases (HVD) are a leading cause of cardiovascular morbidity and mortality. In a recent epidemiological study, it was estimated that 47M people have suffered from HVD worldwide. They affect 8 to 13% of the population aged over 65 years worldwide, with an increasing incidence anticipated with aging population. Here, HVD refer to a group of conditions with the three most prevalent heart valve diseases being aortic stenosis (9M), mitral regurgitation (24 M) and tricuspid regurgitation (10M). HVD represent a major medical and economic burden. The absence of treatment of valve diseases leads to heart failure, increased rehospitalizations which impact on quality of life and significantly the economy of the health system. Costs related to HVD-related heart failure were $30B in 2022 and estimated to be $70B in 2030.

In HVD, mitral and tricuspid heart diseases are now the major unmet needs. Following clinical success of transcatheter aortic valve implantation (TAVI) introduced 20 years ago, innovative transcatheter approaches and devices are emerging to target mitral regurgitation (MR) and tricuspid regurgitation (TR). Although new devices and catheter iterations may help in obtaining optimal mitral or tricuspid Transcatheter Edge to Edge Repair (TEER) results, many patients remain not suitable to TEER, and interventions to correct TR are underutilized in daily clinical practice: less than 1% of TR patients are treated because of increased surgical risk and late patient presentation. More than 50% with severe MR are inoperable or at increased surgical risk. Yet, patient selection/inclusion criteria remain the major challenge to improve outcomes and increase adoption of transcatheter procedures.

Although new device and catheter iterations may help obtaining optimal Mitral Transcatheter Edge to Edge Repair (M-TEER) results, a significant proportion of patients with a complex anatomy might be better addressed by new interventional techniques, including transcatheter mitral valve (MV) replacement instead of M-TEER. By avoiding the morbidity of open mitral replacement and effectively preventing recurrence of MR, transcatheter mitral valve implantation (TMVI) could provide a good option for treating mitral valve disease. Nonetheless, TMVI must face some anatomical challenges that remain the most important factor limiting the eligibility to TMVI. Based on CHOICE-MI registry, the largest real-world TMVI registry, one third of patients referred for TMVI were screen failed for LVOT obstruction risk (FIG. 1). Accordingly, there is a need for improved TMVI methods and devices.

### SUMMARY

Devices, systems, and methods for cutting leaflets in cardiac valves including mitral, aortic, and tricuspid valves are disclosed herein. In some embodiments, a system is designed for accessing and cutting the mitral valve or even accessing and cutting the mitral valve post TEER prior to installation of a transcatheter mitral valve prosthesis. Grabbing the valve may require adaptations such as a high angle navigation of the catheter (up to 180 degrees) and an inverted V-shaped grasper for proper navigation without getting stuck in the chordae. Distal tip navigation, including 2D+ rotation, may be necessary for mitral sectioning. Such a system may also be used for cutting leaflets in the aortic and tricuspid valves.

In some embodiments, the device interfaces to a standard apparatus (e.g. electrocutter, radio frequency (RF)) present in many hospitals, and does not require a separate instrument for the delivery of the cutting energy. The cutting element may be navigated without protection from a sheath. This facilitates delivery and grasping, and simplifies design of the catheter.

### BRIEF DESCRIPTION OF THE DRAWINGS

FIG. 1 shows an illustration of a left ventricular outflow tract (LVOT) obstruction. A transcatheter heart valve positions the anterior mitral leaflet such that the outflow tract is obstructed. The curved arrows show blood flow which is hindered by the LVOT obstruction.

FIG. 2 shows illustrations of the use of the V-grasper to cut mitral and tricuspid valves post-clip procedures.

FIG. 3 shows a graph illustrating US transcatheter aortic valve replacement (TAVR) market forecasts.

FIG. 4A shows an illustration of an example of a catheter control unit.

FIG. 4B shows an illustration of the interior mechanisms of the catheter control unit of FIG. 4A and the cutting source connection.

FIG. 5 shows an exemplary grasper position for insertion in a femoral artery.

FIG. 6 shows an exemplary V-shaped grasper in partial deployment for grasping. The grasper has a straight cutting channel.

FIG. 7 shows the V-shaped grasper of FIG. 5 in an inverted “Mexican hat” configuration.

FIG. 8 shows an illustration of an exemplary device containing both a grasper/cutter, and flexible elements providing for direction control. The cross-sectional view shows the guidewire within the central lumen and additional wires (pull wire steer, guidewire RF, and pull wire inverted V) within the array of lumens surrounding the central lumen.

FIG. 9 shows illustrations demonstrating assembly of the distal end of the exemplary device of FIG. 8. The flexible distal end can bend in one plane more than 180 degrees, and in the perpendicular plane up to 30 degrees.

FIG. 10 shows illustrations of a grasper having dual-cut capabilities. A central channel accommodates a wire for linear cuts while an external U-shaped channel accommodates a second independent wire for U-shaped cuts. Wire selection may be controlled by an operator via the catheter control unit.

FIG. 11 shows illustrations of an exemplary gripper device transitioning from a fully retracted (closed) base configuration to a partially engaged “Mexican hat” configuration and then to a fully engaged gripping configuration. In the fully retracted configuration, the gripper fits within a 15 French (Fr) diameter.

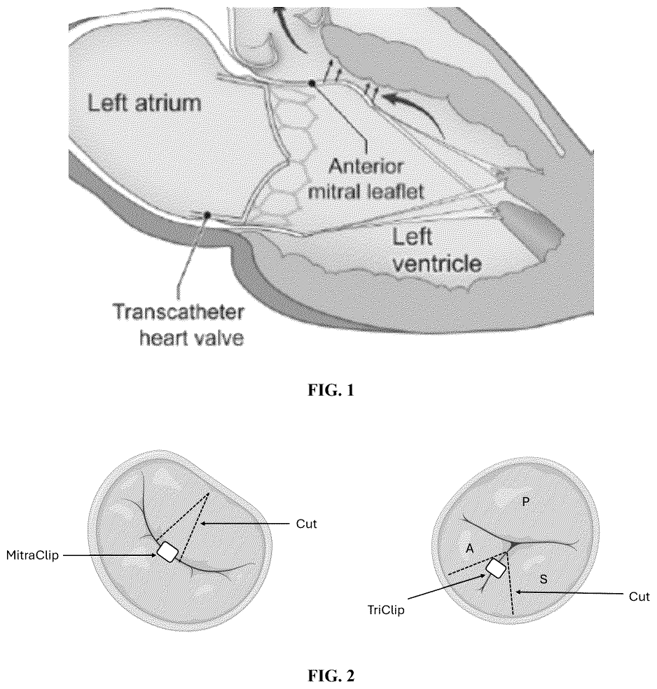

### DETAILED DESCRIPTION

In some embodiments, a device for cutting a leaflet of a mitral, aortic, or tricuspid valve is disclosed. In some embodiments, the device comprises: a catheter having a proximal end and a distal end; and a pair of gripping jaws connected to the distal end of the catheter. In some embodiments, the pair of gripping jaws is configured to actuate from: a fully engaged configuration in which the gripping jaws are substantially parallel to each other; to a fully retracted configuration in which the gripping jaws are separated by an angle greater than 180 degrees. In some embodiments, the angle of separation of the gripping jaws is the angle between gripping faces of the gripping jaws. For example, a fully engaged configuration with gripping faces in contact could have a zero angle of separation and a fully retracted configuration with gripping jaws positioned back-to-back could have a 360 degree angle of separation. In some embodiments, the gripping jaws are separated by an angle greater than 190, 200, 210, 220, 230, 240, 250, 260, or 270 degrees in their fully retracted configuration. In some embodiments, the gripping jaws are separated by an angle of about 360 degrees in their fully retracted configuration.

In some embodiments, the device is for cutting a leaflet of a mitral valve. In some embodiments, the device additionally comprises a cutting apparatus. In some embodiments, the cutting apparatus is an electrocutter. In some embodiments, the cutting apparatus is an optical cutter. In some embodiments, the cutting apparatus is a radio frequency (RF) cautery apparatus. In some embodiments, the cutting apparatus is a laser cutter. In some embodiments, the cutting apparatus is a microwave cutter. In some embodiments, the cutting apparatus is a focused ultrasound cutter. In some embodiments, the cutting apparatus is a pulsed field cutter. In some embodiments, the cutting apparatus is a steam cutter. In some embodiments, the cutting apparatus comprises one or more wires configured for connection with a radio frequency (RF) or electrical energy generator. In some embodiments, the cutting apparatus comprises a U-shape. In some embodiments, the device additionally comprises multiple cutting apparatuses that can be configured by the operator to remove a section of tissue or to perform a linear cut.

In some embodiments, at least one of the gripping jaws comprises a plurality of gripping teeth. In some embodiments, at least one of the gripping jaws is configured to pivot about an attachment point. In some embodiments, each of the gripping jaws is configured to pivot about an attachment point. In some embodiments, at least one of the gripping jaws comprises a cutting channel. In some embodiments, each of the gripping jaws comprises a cutting channel.

In some embodiments, the device additionally comprises a flexible segment between the proximal end and the distal end. In some embodiments, the flexible segment comprises a laser-cut hypotube. In some embodiments, the flexible segment is configured to provide U-turn control to the pair of gripping jaws such that they may be actuated from +180 degrees to −180 degrees relative to the catheter. In some embodiments, the flexible segment is configured to provide lateral control to the pair of gripping jaws such that they may be actuated from +30 degrees to −30 degrees relative to the catheter.

In some embodiments, the device additionally comprises a plurality of gripping pull wires configured for actuation of the pair of gripping jaws. In some embodiments, the device additionally comprises a plurality of control pull wires configured for control of catheter flexing. In some embodiments, the catheter has a main lumen and a plurality of satellite lumens. In some embodiments, the main lumen is configured to surround a guide wire. In some embodiments, the satellite lumens are configured to surround a plurality of control pull wires. In some embodiments, one or more of the satellite lumens are configured to surround an energy carrying wire. In some embodiments, one or more of the satellite lumens are configured to surround an optical fibre.

In some embodiments, the device is configured for insertion over a guide wire. In some embodiments, the device is configured for transfemoral retrograde approach through an aortic valve. In some embodiments, the device is configured for sheath-free insertion. In some embodiments, the device is configured for use with a steerable sheath. In some embodiments, the device is configured for transfemoral retrograde approach using a steerable sheath. In some embodiments, the device is configured for transeptal approach using a steerable sheath.

In some embodiments, the device is configured for navigation from a femoral artery to a left ventricle. In some embodiments, the device is configured for navigation to the mitral valve through a transeptal approach, using a guiding steerable sheath. In some embodiments, the device is configured to avoid entanglement with chordae tendineae. In some embodiments, the device is configured to avoid entanglement with chordae tendineae by navigation in a partially engaged configuration.

In some embodiments, the device is configured for use as part of a transcatheter mitral valve implantation (TMVI). In some embodiments, the device is configured for use as part of a mitral or tricuspid Transcatheter Edge to Edge Repair (TEER) removal. In some embodiments, the device is configured for use as part of a transcatheter aortic valve implantation (TAVI). In some embodiments, the device is configured for use as part of a TAV in surgical bioprosthesis (ViV) or TAV-in-TAV transcatheter aortic valve implantation.

In some embodiments, a system comprising a device of claim 1 and a catheter control unit is disclosed. In some embodiments, the catheter control unit comprises a plurality of control knobs configured for actuation of the device. In some embodiments, the catheter control unit comprises a first control knob for controlling engagement and retraction of the pair of gripping jaws. In some embodiments, the catheter control unit comprises a second control knob for controlling flexing of the catheter to navigate the pair of gripping jaws towards the valve leaflet. In some embodiments, the catheter control unit comprises a third control knob for controlling lateral flexing of the catheter. In some embodiments, the second control know controls flexing of the catheter in a first plane and the third control knob controls flexing of the catheter in a second plane, perpendicular to the first plane. In some embodiments, the catheter control unit comprises a connection for a guidewire. In some embodiments, the catheter control unit comprises a connection for an electrical or radio frequency (RF) generator. In some embodiments, the catheter control unit comprises two connections for an electrical or RF generator. In some embodiments, a first connection to an electrical or RF generator is configured to control straight cuts and a second connection is configured to control U-shaped cuts.

In some embodiments, a method of cutting a leaflet of a mitral, aortic, or tricuspid valve is disclosed. In some embodiments, the method comprises: providing a device comprising: a catheter having a proximal end and a distal end; a pair of gripping jaws connected to the distal end of the catheter, the pair of gripping jaws configured to actuate from: a fully engaged configuration in which the gripping jaws are substantially parallel to each other; to a fully retracted configuration in which the gripping jaws are separated by an angle greater than 180 degrees; navigating the pair of gripping jaws to the valve while the pair of gripping jaws are in the fully retracted configuration; actuating the pair of gripping jaws to the fully engaged configuration such that the pair of gripping jaws grasp a leaflet of the valve; and cutting the leaflet of the valve while the leaflet is grasped by the pair of gripping jaws.

In some embodiments, the valve is a mitral valve. In some embodiments, the method additionally comprises actuating the catheter to flex about 180 degrees prior to actuating the pair of gripping jaws to the fully engaged configuration. In some embodiments, flexing the catheter inverts the pair of gripping jaws to orient the pair of gripping jaws towards the leaflet.

### EXAMPLES

The following examples are put forth so as to provide those of ordinary skill in the art with a complete disclosure and description of how to make and use the present disclosure, and are not intended to limit the scope of what the inventors regard as their disclosure, nor are they intended to represent or imply that the experiments below are all of or the only experiments performed. It will be appreciated by persons skilled in the art that numerous variations and/or modifications may be made to the disclosure as shown in the specific aspects without departing from the spirit or scope of the disclosure as broadly described. The present aspects are, therefore, to be considered in all respects as illustrative and not restrictive.

### Example 1: Leaflet Cutting Device for Mitral, Aortic, or Tricuspid Valves

TMVI inclusion criteria could be expanded by removing the LVOT obstruction risk, for example, by cutting the leaflet of the mitral valve causing the obstruction to free the outflow track. In this example, this is achieved via a catheter that can be navigated from the femoral artery to the left ventricle and then perform a 180 degree turn to orient a grasping mechanism towards the mitral leaflet. The leaflet is then grasped and cut using energy delivery that can be delivered using various mechanisms (electric, RF, optical).

The retro aortic approach is designed to achieve leaflet splitting while the TMVI prosthesis is already in the left atrium. This will avoid the worsening of mitral regurgitation with hemodynamic compromise for a long time, time needed to advance the delivery system through the inferior vena cava, cross the atrial septum and steer to the left atrium. One primary challenge in performing this maneuver and a distinguishing feature of this invention is that the catheter must navigate through the chordae: a standard closed tip/grasping catheter will get entangled in the chordae rendering the procedure difficult as it is navigated back from the bottom of the left ventricle up to the site of the mitral valve cut. This exemplary device comprises an inverted V-shaped grasper and catheter to avoid chordae in mitral sectioning applications.

The design of this exemplary device was guided by drawbacks of prior experimental designs. In a previous prototype, the navigation mechanism integrated the energized wire in the same guide as the one used for the guiding wire, making in vivo applications unfeasible. Furthermore, the wires used for navigation were not guided inside the catheter leading to unwanted pull-forces and poor directionality. Finally, the grasping unit only opened to a maximum of 240 degrees. In this exemplary design, the opening angle range is expanded to a full inverted position facilitating insertion and navigation of the catheter in the inverted position, and the ensuing grasping movement. The catheter and associated V-grasper can form an inverted ‘V,’ or a “Mexican Hat” shape to navigate the tip up without getting stuck in small cords that connect the leaflet to the papillary muscle. While in this shape, the grasper's position is inverted. Having two mobile jaws that can open past 180 degrees can facilitate and stabilize the grasping of the anterior mitral leaflet.

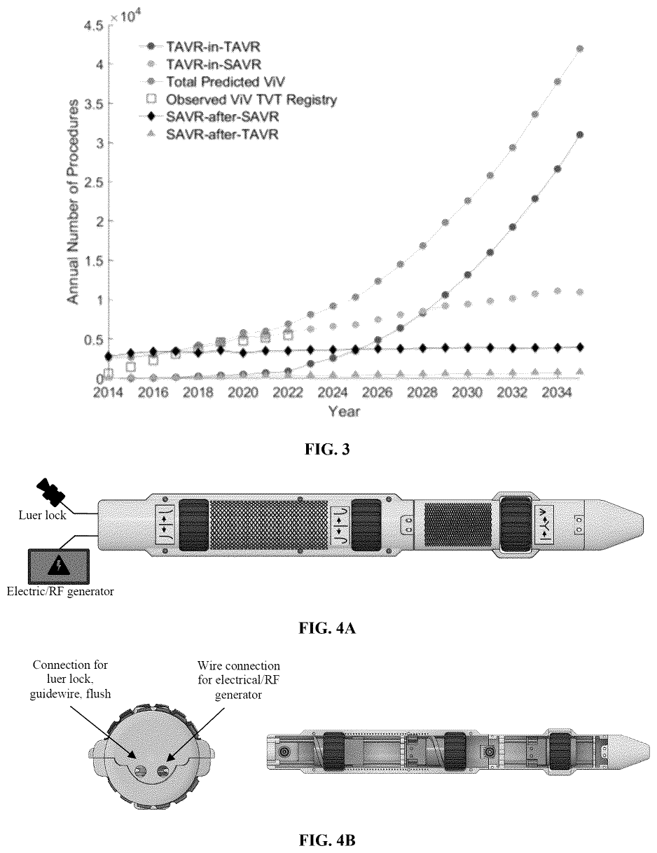

Other applications of the exemplary device include cutting the leaflet after TEER procedures to enable the installation of a replacement valve, both in mitral and tricuspid positions (FIG. 2). The TRILUMINATE (NCT #03904147) trial showed that 13% of patients remained with severe or massive tricuspid regurgitation (TR) at 30 days and if adding other transcatheter repair solutions were not possible, only a transcatheter replacement solution can address the residual TR. This can only be done after cutting the leaflet bridge by a dedicated transcatheter solution. Parallelly, patients with residual mitral regurgitation (MR) after M-TEER, especially those with high trans mitral gradient will need TMVI after cutting the leaflet bridge. The device can also be used in the aortic setting, both for native and prosthetic valves. A bicuspid valve with bulky calcified leaflet will need to split the common leaflet to avoid coronary occlusion. In the US, the annual number of valve-in-valve (ViV) procedures (TAV in SAVR and TAV in TAV) is expected to reach 42,000 procedures in 2035 (15%) of all TAVR (FIG. 3). Without splitting leaflet, 25% of patient with surgical aortic valve replacement (SAVR), 40% patients with index S3 and 60% of patients with index EVOLUT valves are not eligible for ViV procedures.

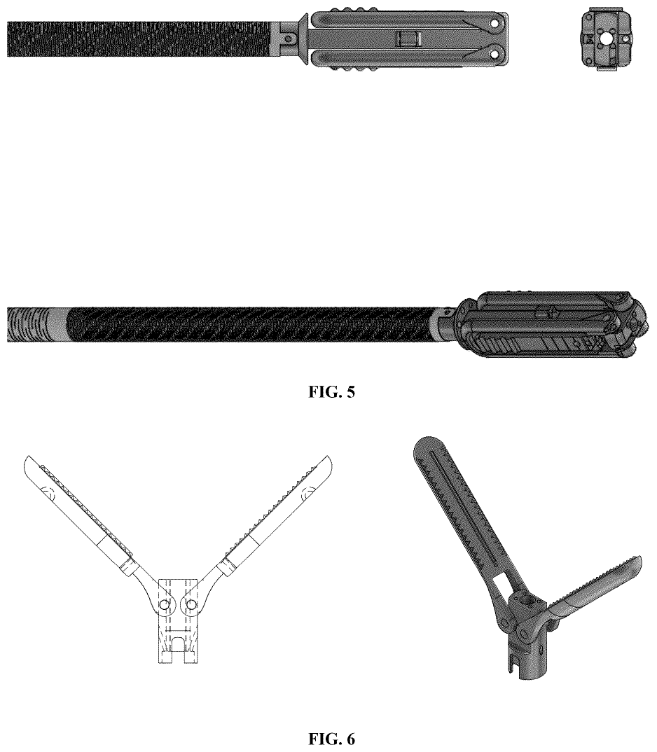

A first embodiment of the catheter is represented in FIG. 4A and FIG. 4B, where a control handle, containing three knobs to control the direction of the distal end (2 directions+rotation of the catheter for orientation), and third knob to control engagement and retraction of the V-grasper. The directional extremity is designed to accommodate a full 180-degree movement in the left ventricle (U-turn) to enable transfemoral access to the mitral valve and grasping of the leaflet to cut. The lateral control is used to select the positioning of the cut on the valve leaflet. In addition to the control provided by the knobs, in some embodiments, rotation of the catheter provides control of the gripping jaws. For example, in some embodiments, clockwise rotation of the catheter is configured to move the gripping jaws in an anterior direction and counterclockwise rotation of the catheter is configured to move the gripping jaws in a posterior direction.

The grasper can allow inversion of the cutting system to form a 180-degree flat surface, to facilitate mitral and tricuspid valve capture and also to invert beyond 180 degrees, (to 240 degrees, the so-called “Mexican Hat” position and to 360 degrees for the initial inverted mode to facilitate the insertion of the catheter in the femoral artery) enabling the system to navigate through the chordae and avoid entanglement. A guide wire is inserted in the proximal end and exits at the cusp of the V system (FIG. 5).

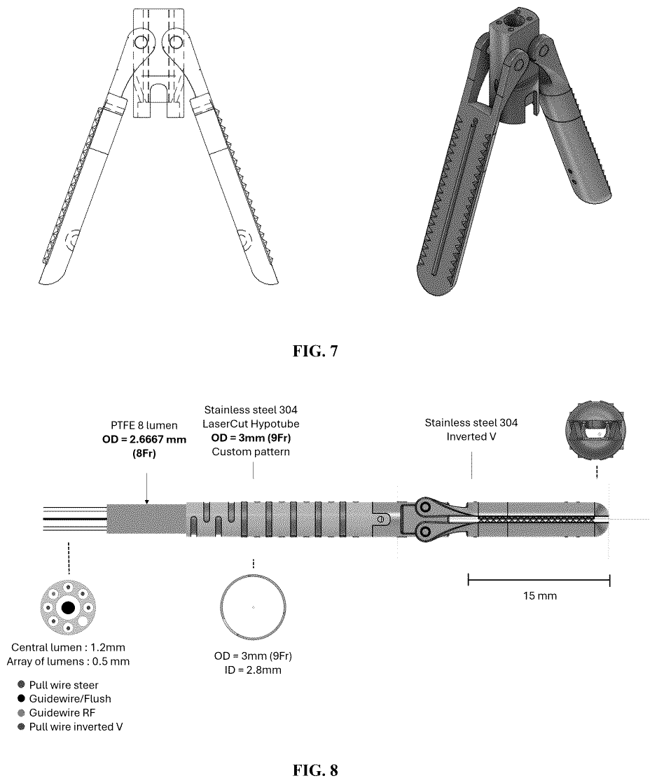

This exemplary cutting device contains no blade but can accommodate an energy delivery system (e.g. a conducting wire which is fixed to the grasper for electrocuting tissue, a RF delivery device or fiber optics for optical energy delivery to cut the tissue). An implementation of the cutting device represented in FIG. 6 and FIG. 7 used a single wire and lacks large teeth which could be an impediment to chordae navigation. Other configurations are also possible. For example, the device can implement a U-shaped cutter for removal of a larger portion of the leaflet or a cutter with an RF-optimized probe (RF has been documented to accelerate coagulation post-cutting compared to alternatives). The jaws can be controlled with pull wires.

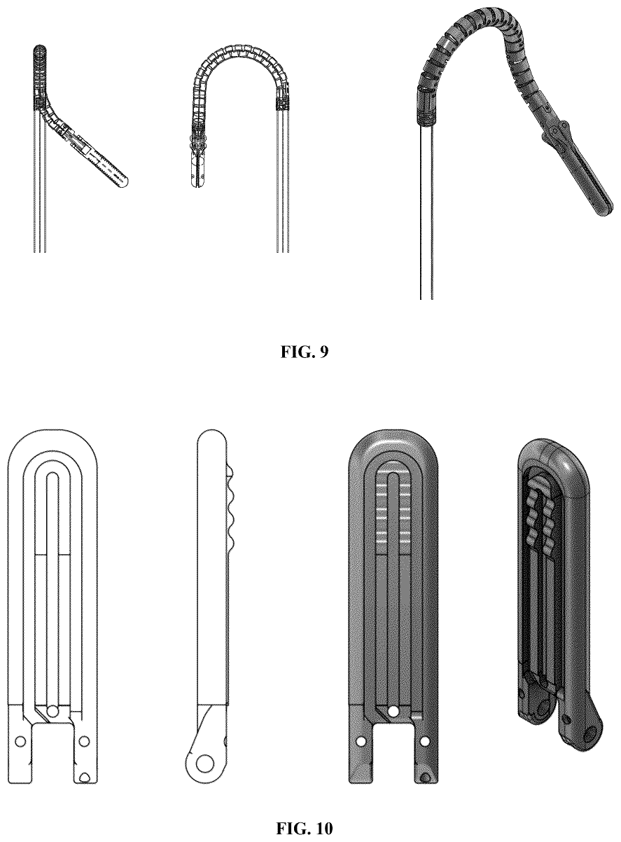

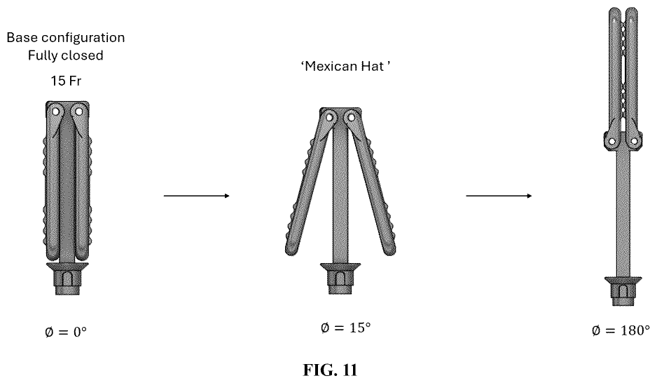

This exemplary device contains both grasper/cutter and flexible elements for direction control (FIG. 8 and FIG. 9). The shaft is composed of a multi-lumen polytetrafluoroethylene (PTFE) tube where guidewire and flush go through the main lumen, and pull wires and wire for RF, electrical or optical fiber cutting go through the satellite lumens. This allows guiding the large number of wires without intersection of tangles. Instead of a sheath, a custom stainless-steel laser cut hypotube with variables cut patterns for proximal and distal end is placed around the PTFE tube. The flexible distal end can bend in one plane more than 180 degrees, and in the perpendicular plane up to 30 degrees.

This exemplary catheter design delivers multiple key aspects to functionality, including: full grasping inversion and inverted navigation, guiding the large number of wires for direction control, guide-wire follow-up, and simplified design with integrated multi-lumen PTFE tubes and laser-cut hypotubes.

### Example 2: Use of Leaflet Cutting Device as Part of a Transcatheter Mitral Valve Implantation (TMVI)

Step 1: Place an introducer for femoral artery access and insert guide wire all the way to the left ventricle.

Step 2: Insert catheter in the introducer and over the guide wire, move the catheter to the left ventricle.

Step 3: Remove guide wire and navigate catheter to the mitral valve by performing a 180 degree turn at the base of the papillary muscles while using the grasper in inverted form to avoid being caught in chordae. Grasp the mitral valve leaflet and activate energy for cutting.

While this disclosure is satisfied by embodiments in many different forms, as described in detail in connection with preferred embodiments of the disclosure, it is understood that the present disclosure is to be considered as exemplary of the principles of the disclosure and is not intended to limit the disclosure to the specific embodiments illustrated and described herein. Numerous variations may be made by persons skilled in the art without departure from the spirit of the disclosure. The scope of the disclosure will be measured by the appended claims and their equivalents. The abstract and the title are not to be construed as limiting the scope of the present disclosure, as their purpose is to enable the appropriate authorities, as well as the general public, to quickly determine the general nature of the disclosure. In the claims that follow, unless the term “means” is used, none of the features or elements recited therein should be construed as means-plus-function limitations pursuant to 35 U.S.C. § 112, ¶6.

1. A device for cutting a leaflet of a mitral, aortic, or tricuspid valve, the device comprising:
a. a catheter having a proximal end and a distal end; and
b. a pair of gripping jaws connected to the distal end of the catheter such that the device is configured to navigate the pair of gripping jaws to the valve leaflet avoiding chordae entanglement,
the pair of gripping jaws configured to actuate from:
a fully engaged configuration in which the gripping jaws are substantially parallel to each other; to
a fully retracted configuration in which the gripping jaws are separated by an angle greater than 180 degrees.

2. The device of claim 1, wherein the gripping jaws are separated by an angle greater than 190, 200, 210, 220, 230, 240, 250, 260, or 270 degrees in their fully retracted configuration.

3. The device of claim 1, wherein the gripping jaws are separated by an angle of about 360 degrees in their fully retracted configuration.

4. The device of claim 1, wherein the device is configured to navigate the pair of gripping jaws to the valve leaflet using a catheter control unit.

5. The device of claim 1, wherein the device is configured to navigate the pair of gripping jaws to the valve leaflet such that the pair of gripping jaws is oriented toward the valve leaflet.

6. The device of claim 1, wherein the pair of gripping jaws is configured to avoid chordae entanglement during navigation to the valve leaflet by navigation in a partially engaged configuration.

7. The device of claim 1, wherein the device is for cutting a leaflet of a mitral valve.

8. The device of claim 1, wherein the device additionally comprises a cutting apparatus.

9. The device of claim 8 wherein the cutting apparatus is an electro-cutter.

10. The device of claim 8 wherein the cutting apparatus is an optical cutter.

11. The device of claim 8 wherein the cutting apparatus is a radio frequency (RF) cautery apparatus.

12. The device of claim 8, wherein the cutting apparatus is a laser cutter.

13. The device of claim 8, wherein the cutting apparatus is a microwave cutter.

14. The device of claim 8, wherein the cutting apparatus is a focused ultrasound cutter.

15. The device of claim 8, wherein the cutting apparatus is a pulsed field cutter.

16. The device of claim 8, wherein the cutting apparatus is a steam cutter.

17. The device of claim 8, wherein the cutting apparatus comprises one or more wires configured for connection with a radio frequency (RF) or electrical energy generator.

18. The device of claim 8, wherein the cutting apparatus comprises a U-shape.

19. The device of claim 8, wherein the cutting apparatus is configured to perform a linear cut.

20. The device of claim 1, wherein the device additionally comprises multiple cutting apparatuses configured to remove a section of tissue or to perform a linear cut.

21. The device of claim 1, wherein at least one of the gripping jaws comprises a plurality of gripping teeth.

22. The device of claim 1, wherein at least one of the gripping jaws is configured to pivot about an attachment point.

23. The device of claim 1, wherein each of the gripping jaws is configured to pivot about an attachment point.

24. The device of claim 1, wherein at least one of the gripping jaws comprises a cutting channel.

25. The device of claim 1, wherein each of the gripping jaws comprises a cutting channel.

26. The device of claim 1, additionally comprising a flexible segment between the proximal end and the distal end.

27. The device of claim 26, wherein the flexible segment comprises a laser-cut hypotube.

28. The device of claim 26, wherein the flexible segment is configured to provide U-turn control to the pair of gripping jaws such that they may be actuated from +180 degrees to −180 degrees relative to the catheter.

29. The device of claim 26, wherein the flexible segment is configured to provide lateral control to the pair of gripping jaws such that they may be actuated from +30 degrees to −30 degrees relative to the catheter.

30. The device of claim 1, wherein rotation of the catheter provides control of the gripping jaws.

31. The device of claim 1, wherein clockwise rotation of the catheter is configured to move the gripping jaws in an anterior direction and counterclockwise rotation of the catheter is configured to move the gripping jaws in a posterior direction.

32. The device of claim 1, additionally comprising a plurality of gripping pull wires configured for actuation of the pair of gripping jaws.

33. The device of claim 1, additionally comprising a plurality of control pull wires configured for control of catheter flexing.

34. The device of claim 1, wherein the catheter has a main lumen and a plurality of satellite lumens.

35. The device of claim 34, wherein the main lumen is configured to surround a guide wire.

36. The device of claim 34, wherein the satellite lumens are configured to surround a plurality of control pull wires.

37. The device of claim 34, wherein one or more of the satellite lumens are configured to surround an energy carrying wire.

38. The device of claim 34, wherein one or more of the satellite lumens are configured to surround an optical fiber.

39. The device of claim 1, wherein the device is configured for insertion over a guide wire.

40. The device of claim 1, wherein the device is configured for transfemoral retrograde approach through an aortic valve.

41. The device of claim 1, wherein the device is configured for use with a steerable sheath.

42. The device of claim 1, wherein the device is configured for transeptal approach.

43. The device of claim 1, wherein the device is configured for transfemoral retrograde approach.

44. The device of claim 1, wherein the device is configured for navigation from a femoral artery to a left ventricle.

45. The device of claim 1, wherein the device is configured for navigation to the mitral valve through a transeptal approach, using a guiding steerable sheath.

46. The device of claim 1, wherein the device is configured to avoid entanglement with chordae tendineae.

47. The device of claim 1, wherein the device is configured for use as part of a transcatheter mitral valve implantation (TMVI).

48. The device of claim 1, wherein the device is configured for use as part of a mitral or tricuspid Transcatheter Edge to Edge Repair (TEER) removal.

49. The device of claim 1, wherein the device is configured for use as part of a transcatheter aortic valve implantation (TAVI).

50. The device of claim 1, wherein the device is configured for use as part of a TAV in a surgical bioprosthesis (ViV).

51. The device of claim 1, wherein the device is configured for use as part of a TAV-in-TAV transcatheter aortic valve implantation.

52. A system comprising a device of claim 1 and a catheter control unit.

53. The system of claim 52, wherein the catheter control unit comprises a plurality of control knobs configured for actuation of the device.

54. The system of claim 52, wherein the catheter control unit comprises a first control knob for controlling engagement and retraction of the pair of gripping jaws.

55. The system of claim 52, wherein the catheter control unit comprises a second control knob for controlling flexing of the catheter to invert the pair of gripping jaws.

56. The system of claim 52, wherein the catheter control unit comprises a third control knob for controlling lateral flexing of the catheter.

57. The system of claim 52, wherein the catheter control unit comprises a connection for a guidewire.

58. The system of claim 52, wherein the catheter control unit comprises a connection for an electrical or radio frequency (RF) generator.

59. A method of cutting a leaflet of a mitral, aortic, or tricuspid valve, the method comprising:
a. providing a device comprising:
i. a catheter having a proximal end and a distal end; and
ii. a pair of gripping jaws connected to the distal end of the catheter, the pair of gripping jaws configured to actuate from: a fully engaged configuration in which the gripping jaws are substantially parallel to each other; to a fully retracted configuration in which the gripping jaws are separated by an angle greater than 180 degrees;
b. navigating the pair of gripping jaws to the valve while the pair of gripping jaws are in the fully retracted configuration;
c. actuating the pair of gripping jaws to the fully engaged configuration such that the pair of gripping jaws grasp a leaflet of the valve; and
d. cutting the leaflet of the valve while the leaflet is grasped by the pair of gripping jaws.

60. The method of claim 59, wherein the valve is a mitral valve.

61. The method of claim 59, additionally comprising actuating the catheter to flex about 180 degrees prior to actuating the pair of gripping jaws to the fully engaged configuration.

62. The method of claim 59, wherein flexing the catheter inverts the pair of gripping jaws to orient the pair of gripping jaws towards the leaflet.
